(19) United States
(12) Patent Application Publication
(10) Pub. No.: US 2010/0292699 A1
(43) Pub. Date: Nov. 18, 2010

(54) Surgical Milling Cutter and Blades for Such a Cutter

(76) Inventor: Marc-Etienne Favre, Belmont-sur-Lausanne (CH)

Correspondence Address: BACON & THOMAS, PLLC, 625 SLATERS LANE, FOURTH FLOOR, ALEXANDRIA, VA, 22314-1176, US

(21) Appl. No.: 12/087,005

(22) PCT Filed: Oct. 9, 2006
(86) PCT No.: PCT/CH2006/000558

§ 371 Date: Jun. 24, 2008

(30) Foreign Application Priority Data
Dec. 28, 2005 (CH) 2085/05

(51) Int. Cl. A61B 17/16

(52) U.S. Cl. 606/80

(57) ABSTRACT

This milling cutter is composed of four blades (Ia, 1b, 1c, 1d) cut out, preferably by die-stamping, from sheets of soft metal, for example stainless steel. The blades have slits (13, 15) that allow them to be inserted into one another in such a way as to obtain a cutter head. The slits have different widths, which allows them to be assembled on the rotation shaft in a number greater than two. Each blade is continued by a lower part (12). These lower parts are engaged in a flat ring (7) that keeps the assembly rigid. Two of the blades each have two lateral extensions (2) that are cut out from the blade itself and are situated in the lower part (12) of the blade. These lateral extensions are designed in such a way as to permit their insertion in notches (3) of conventional bayonet-type cutter spindles, thereby avoiding the need for an intermediate tool.

### TECHNICAL FIELD

This invention relates to a surgical reamer and, more specifically, to an acetabular reamer, intended for reaming the hip bone for the purpose of inserting a prosthesis.

### BACKGROUND ART

Prior art reamers, for use in medical applications, and particularly acetubular reamers, are shaped like a hemispherical cap, with openings and cutting edges, similar to those of cheese graters; such reamers are formed by stamping, blanking and spinning. Examples of such hemispherical reamers may be found in documents CH 692600, CH 690021, FR 2281095, U.S. Pat. No. 4,811,632, and U.S. Pat. No. 5,100,267.

Reamers of the type mentioned above are relatively expensive to produce, mainly because of the various operations involved in this kind of manufacture.

In the medical field, particularly that of acetubular reamers, the cost prevents users from using them only once, whereas single usage would be far preferable because of septic risks.

The document U.S. Pat. No. 5,100,267 describes a disposable acetubular reamer cup. In order to reduce its price, the acetabular reamer described in that document is fitted with a polymeric plug, which is easy to make and inexpensive. However, the cutting bowl, in stainless steel, must be manufactured by conventional means. This means that it remains expensive. Moreover, the presence of plastic matter often presents risks, since this type of very soft material can easily leave particles in the body, which often are not well tolerated by the patient.

Consequently, medical practitioners continue to use acetabular reamers, usually free of any plastic material, that are expensive and that they must sterilise after each use, which involves relatively substantial costs.

Moreover, reamers are of solid construction, such that the reamer has no interstitial space that allows the user to view the bottom of the cavity hollowed out by the reamer. The user therefore is hardly able to check the progress of the reaming during the operation, or the state of the reamed cavity walls. This is a serious inconvenience particularly in the medical field.

Other reamers have cutting blades which are radially disposed, equiangularly spaced around the axis of rotation, and which extend outwards. Such examples may be found in patents FR1041311, FR1031888, U.S. Pat. No. 3,702,611 or U.S. Pat. No. 4,621,637.

In these examples, the blades are built-up, i.e. manufactured independently and then fixed in the cutter head, which contains housings for this purpose. Means of attachment are required (such as screws or plugs), which complicate the device.

This type of reamer has the same inconveniences as mentioned above, and particularly the difficulty experienced by the surgeon in viewing the bottom of the cavity cut by the reamer.

Moreover, such reamers are still expensive to manufacture.

PCT application No. WO98/31291 shows a relatively simple acetabular reamer, in which flat blades are stamped in a semi-circular shape from sheet metal and are assembled together by means of slots in the blades themselves. The blades cross each other at right angles and are semi-circular. To attach them on to the tool and hold them together, the blades are inserted by means of projections at both ends of each blade into apertures in a base plate. This plate has a central aperture which allows it to be fixed on to a rotating tool.

This reamer model still has the inconvenience of reduced visibility, due to the presence of the base plate. There is also the question of whether the reamer is sufficiently rigid.

PCT application No. WO02/49516 shows another surgical reamer formed from blades stamped from sheet metal and fitted together by means of slots. There are only two blades, which join at the axis of rotation, the mounting slots also being on this axis.

In this reamer, visibility is much better, the surgeon being able to see through the blades to the bottom of the cavity cut by the reamer.

The reamer in question, however, is not very rigid, despite the depositor's affirmation. Moreover, reaming efficiency is probably affected due to the fact that there are only two blades.

Furthermore, the device covered by application PCT WO02/49516 mainly deals with a bayonet-type system for fixing the reamer, comprising stamped blades, on a prior art cutter spindle. Such bayonet-type cutter spindles are described notably in patents EP 0 704 191, EP 0 947 170 and EP 1 129 667. They consist essentially of a shank capped with a cylindrical head with four slots open towards the top, which extend laterally into the wall of the head, and a locking part sliding along the shank equipped with vertical fingers which permit the slots to be closed once the reamer is in place.

However, the reamer described in application PCT WO 02/49516 cannot be fixed directly in the cutter spindle in question. In order to enable such fixation, application PCT WO 02/49516 describes an intermediate tool, in which the reamer is fixed by means of a ball device which, at the bottom end, has a fixing cross made up of two cylindrical parts forming a cross, placed horizontally, each branch of which can be engaged in one of the four slots in the cutter spindle.

The advantage of such a device is that it enables the fixing of a reamer consisting of blades cut from sheet metal on a widely available prior art toolholder, the advantage of this type of reamer being its relatively low cost and the fact that it allows the surgeon to see the cavity cut during the operation.

The invention aims to permit the fixing of this type of reamer directly on a bayonet-type tool, such as described by example in patents EP 0 704 191 and EP 1 129 667, without the need for an intermediate tool.

The present invention also proposes to provide a reamer, permitting such a fixation, which is composed of blades assembled together by means of slots wherein the longitudinal axis of symmetry is preferably coincident with the axis of rotation, there being more than two such blades (to ensure better rigidity) and preferably at least four such blades. Such a reamer, which is easy to manufacture, will be more rigid and will cut more efficiently than prior art reamers, while ensuring optimum visibility during the operation. Moreover, it is less costly to manufacture, since the blades are all stamped from metal sheets.

The invention also intends to provide blades that would allow such construction.

### DISCLOSURE OF THE INVENTION

Generally, the surgical reamer which is the subject of the invention consists of several flat blades assembled together by means of slots. Preferably, the longitudinal axis of symmetry is coincident with the axis of rotation of the reamer; the reamer comprises at least four blades; in the preferred embodiment of the invention, one or several slots of said blades, or one or several sections of said slots, are of a different width from the other slots and/or sections of slots. At least one of the blades has at least one lateral extension cut out from the blade itself and capable of being engaged in a slot of a bayonet-type cutter spindle, notably a bayonet-type cutter spindle of the type described in patents EP 0 704 191 and EP 1 129 667.

In its wider definition, the surgical reamer according to the invention is formed from several flat blades assembled together by means of slots, and at least one of the blades has at least one lateral extension cut out from the blade itself and capable of being engaged into a slot of a bayonet-type cutter spindle.

In a more particular embodiment, at least one of the slots and/or sections of the said slot or slots has a different width from that of at least one other slot and/or section of a slot.

In a more particular embodiment, the longitudinal axis of symmetry of the slots is coincident with the axis of rotation of the reamer.

In a more particular embodiment, the lateral extension has a part which is folded in relation to the plane of the blade in which it is cut.

In a more particular embodiment, the folded part has a rounded end.

In a more particular embodiment, the lateral extension has a cutout.

In a more particular embodiment, the reamer comprises at least one rigidifying element which partially encircles and holds together the cutting blades making up the reamer.

In a more particular embodiment, the rigidifying element is a flat ring wherein the plane is perpendicular to the axis of rotation of the reamer, and which has radial slits in which the blades are engaged.

In a more particular embodiment, the radial slits are made on the inside edge of the flat ring.

In a more particular embodiment, the reamer comprises at least four blades, at least two of which each have two lateral extensions.

In a more particular embodiment, which includes by reference all the others, and which consists of the preferred embodiment of the invention, the reamer comprises four blades, wherein a first blade has at least one slot which runs from the lower edge of said blade, wherein a second blade has at least one slot running from the top of the rounded part of the blade, and each of said slots has, at least along one section, a width corresponding to the thickness of the blade containing the other slot, wherein a third blade has at least one upper slot running from the top of the said third blade, and at least one lower slot running from the lower edge of the said third blade, the width of the upper slot being greater that the width of the lower slot, and wherein the fourth blade has at least one slot running from the top of the said blade, the said slot comprises at lease one outer section and one inner section, the outer section being greater than the inner section, the width of the lower slot of the third blade corresponds to the thickness of the fourth blade, the width of the inner section of the slot of the fourth blade corresponds to the thickness of the third blade, and the width of the upper slot of the third blade and the width of the outer section of the fourth blade are the same and are sized such that once fitted together, the two first blades can be engaged into said upper slot of the third blade (1c) and into the outer section of the fourth blade, the third and fourth blades being themselves fitted together.

In a more particular embodiment, the slot (15) of the first blade has at least two sections of different widths, the outer section being wider than the inner section

In a more particular embodiment, the second blade has at least one lower slot running from its lower edge, the said lower slot being wider than the slot that runs from the top of the blade.

Each of the more particular embodiments of the invention may be applied to the others.

With respect to the blade according to the invention intended for a reamer according to the invention, it comprises a rounded upper part, and a narrower lower part in which is arranged the lateral extension that permits the fixation of the reamer directly into the notch in the bayonet-type cutter spindle.

In a particular embodiment, the blade comprises at least one slot with sections of different widths.

In a more particular embodiment, the blade comprises at least one slot running from the top of the rounded part, or from the lower edge of the blade.

In a more particular embodiment, the blade comprises an upper slot running from the top of the rounded upper part and/or a lower slot running from the lower edge, the width of the upper slot being different from the width of the lower slot.

In a more particular embodiment, the blade comprises an upper slot running from the top of the rounded upper part and/or a lower slot running from the lower edge, at least one of the said upper or lower slots comprising at least one outer section and one inner section, the width of the outer section being greater than the width of the inner section.

Each particular embodiment is applicable to the others.

### BRIEF DESCRIPTION OF THE DRAWINGS

FIG. 1 is an elevation view of a bayonet-type cutter spindle with its reamer in the form of a hemispherical cap, in the prior art technique described in patent EP 0 704 191.

The notches in the bayonet-type device are closed here by the fingers of the sliding locking part, which is pressed against the head.

FIG. 1 is an elevation view of a bayonet-type cutter spindle with its reamer in the form of a hemispherical cap, in the prior art technique described in patent EP 1 129 667.

The sliding locking part has been pulled down and the fingers it carries have been removed from the head, leaving the notches open.

FIG. 3 shows a cavalier perspective of a reamer according to the invention wherein the reamer has four blades, two of which are each fitted with two folded fixing extensions with a rounded end.

FIG. 4 is a cavalier perspective of a reamer in the same embodiment as that in FIG. 3, in the process of being fixed on to the cutter spindle, or being removed from it, in an intermediate position, the fixing extensions of the blades not being at the bottom of the notches designed to house them. The locking fingers are not represented here, to give a clearer view.

FIG. 5 is a detailed view designated A in FIG. 4 and enlarged here, giving a better view of the intermediate position of the fixing extension of the blade in the notch in the cutter spindle head.

FIG. 6 is a front view of a reamer blade in the embodiment in FIGS. 3 to 4, with two fixing extensions, before they were folded.

FIG. 7 is a detailed view designated B in FIG. 6 and enlarged here to give a more precise view of a fixing extension with the fold along which this extension must be folded.

FIG. 8 shows a cavalier perspective of a reamer according to the invention wherein the reamer has four blades, two of which are each fitted with two folded fixing extensions, as in FIG. 3, but wherein the fold along which the ends of the said fixing extensions must be folded is horizontal instead of vertical, the end of the extensions not being rounded.

FIG. 9 is a cavalier perspective of a reamer in the same embodiment as that of FIG. 8, fixed on to the cutter spindle, the fixing extensions of the blades being at the bottom of the notches designed to house them. The locking fingers which lock the reamer in this position are not represented here, to give a clearer view.

FIG. 10 is a detailed view designated by C in FIG. 9 and enlarged here, giving a better view of the position of the fixing extension of the blade in the notch in the cutter spindle head, still without representation of the locking finger.

FIG. 11 is a front view of a reamer blade in the embodiment in FIGS. 8 to 10, with two fixing extensions, before they are folded.

FIG. 12 is a detailed view designated D in FIG. 11 and enlarged here to give a more precise view of a fixing extension with the fold along which this extension must be folded.

FIG. 13 is a cavalier perspective of various components of a reamer in the embodiment in FIGS. 3 to 7, before assembly, and without the rigidifying element.

FIG. 14 is a front view of a flat ring intended to provide rigidity for the unit formed by the blades once assembled.

FIG. 15 is a three-quarter view of a reamer in an embodiment similar to that of FIGS. 3 to 7 and 13, but wherein the blades are hollow at the centre.

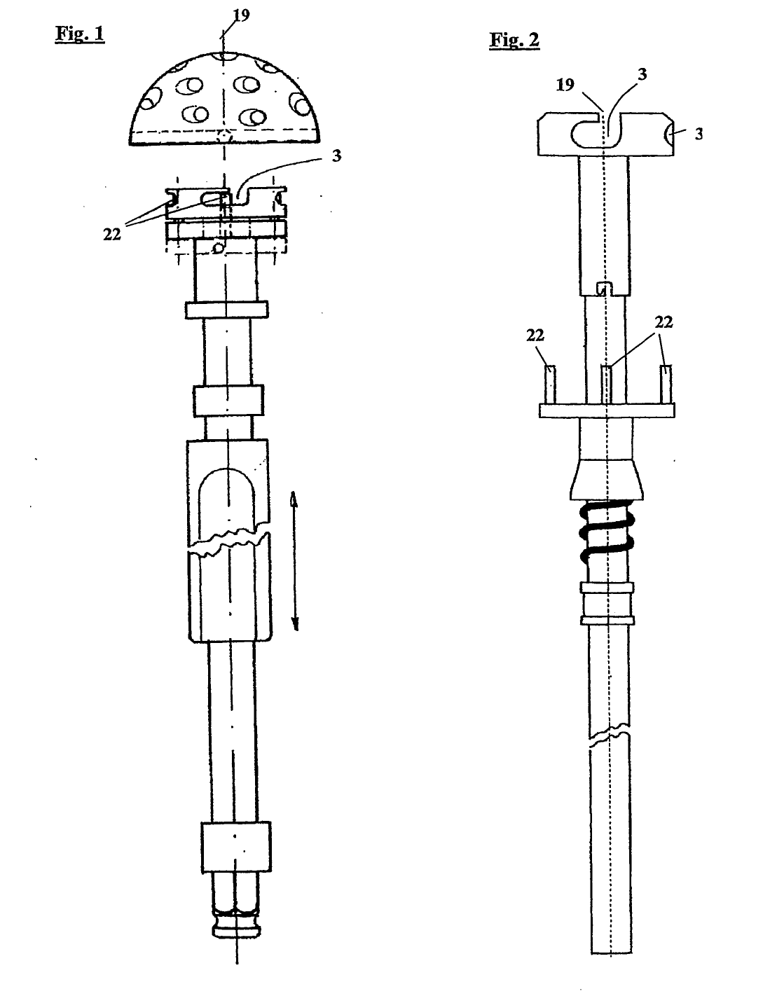

### BEST MODE FOR CARRYING OUT THE INVENTION

An important characteristic of the invention is the fact that the blades may be cut from sheet metal. The term “sheet metal” is not understood to include hard, carbide type, metal sheet, but simply metal sheets that can be stamped, such as stainless steel. The blades 1 of the reamer according to the invention are cut from sheet metal; in the preferred form of the invention, the blades are stamped. This method has the advantage of being very low cost. Laser cutting or spark erosion machining is also possible, but at a less advantageous price.

Preferably, the same operation that stamps the blade will also give it a sharp cutting-edge. It is obviously possible, however, to obtain a cutting-edge by means of a conventional grinding operation.

Likewise, and preferably, the teeth around the edge of the blade as well as any angle on said blades in relation to the plane of the blade, may be obtained by stamping, in a single operation. Experience shows, however, that such an angle is not essential.

In the preferred embodiment of the invention, the reamer is made up of four blades 1a, 1b, 1c and 1d. Each blade has a longitudinal axis of symmetry 11 coincident with the axis of rotation of the reamer 19. The blade edge, i.e. the cutting edge, is rounded, and has a semi-circular shape here. This shape is particularly well adapted to acetabular reamers. It is obvious, however, that numerous other shapes may be used, depending on the intended purposes of the reamer. Each blade has at least one slot 13 or 15, which is on the axis of rotation 19. This longitudinal slot allows the blades to be inserted into one another.

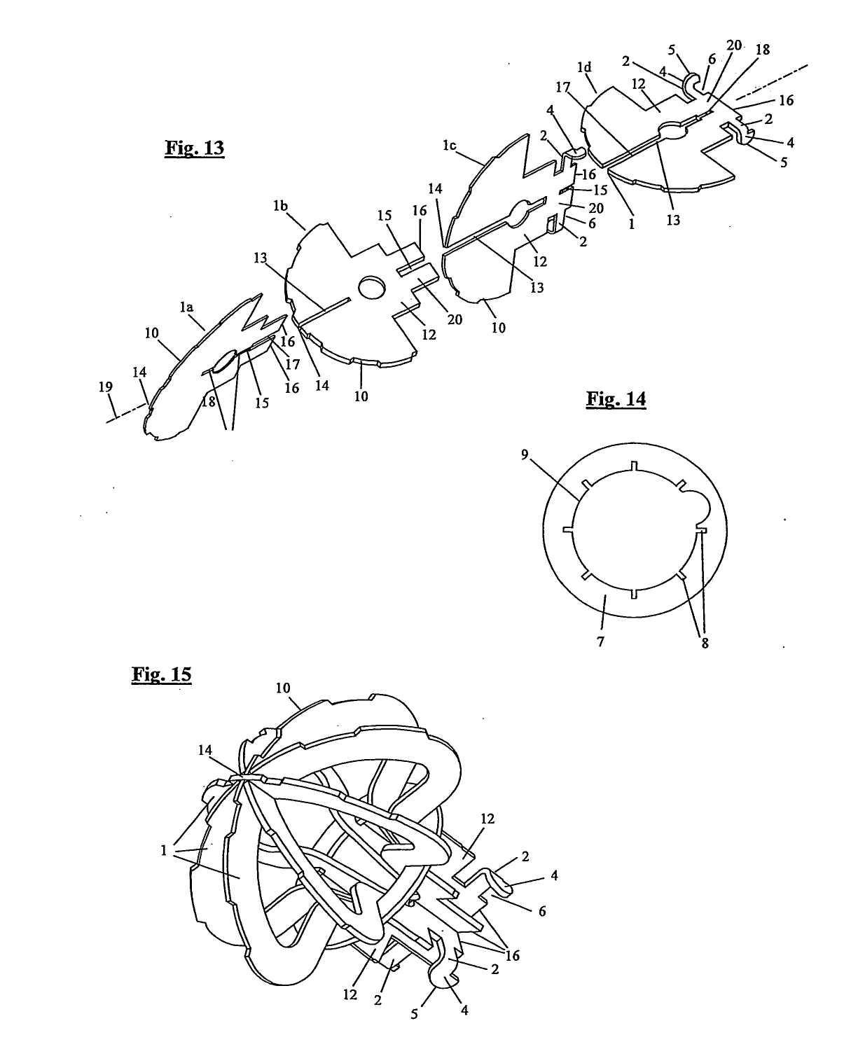

Each blade also had a lower part 12, which here has the shape of a double rectangle that extends the blade in the direction of the cutter spindle head on to which the reamer must be fixed. It is this lower part 12 which enables this assembly. It may be seen in FIG. 13 that the slot 15 in the first blade la runs from the edge 16 of the lower part 12 to halfway up the blade. By contrast, the slot 13 in the second blade 1b runs from halfway up the blade to the top 14 of the blade. In this way, the first two blades fit into each other at right angles. The third blade 1c and the fourth blade 1d each have a slot 13 which runs from their top 14 into their lower part 12. The slot 13 in the third blade 1c is wider than the upper slot 13 of the second blade 1b. The upper part, i.e. the outer section 17 of the slot 13 in the fourth blade 1d likewise is wider. The third blade 1c also has a lower slot 15 which runs from its lower edge 16 to a quarter of the way up said lower part; this lower slot 15 is narrower than the upper slot 13. Its width corresponds to the thickness of the fourth blade 1d. The slot 13 in the fourth blade 1d has two sections 17 and 18; the outer section 17, which runs from the top 14 of the fourth blade 1d to about halfway up the lower part 12, is wider than the inner section 18. The width of the inner section 18 corresponds to the thickness of the third blade 1c. Accordingly, the third and fourth blades can be fitted together, the third blade being inserted in the inner section 18 of the fourth blade 1d and the fourth blade 1d being inserted in the lower slot 15 of the third blade 1c.

The third blade 1c and the fourth blade 1d here are joined to each other by the lower slot 15 and by the third blade and by the inner section 18 of the fourth blade 1d, at right angles.

The group formed by the first two blades 1a and 1b is then inserted into the upper slot 13 and the outer section 17 of the group formed by the third and fourth blades.

In the embodiments represented in the drawings, the slot 15 of the first blade 1a has an inner section 18 and an outer section 17, with the latter extending into the bottom 20 of the lower part 12.

This outer section 17 is wider than the inner section 18.

Likewise, the second blade 1b has a lower slot 15 also situated in the bottom 20 of the lower part 12. The greater width of this lower slot 15 of the second blade 1b and of the outer section 17 of the slot 13 of the first blade 1a permits the insertion of the assembly formed by the third and fourth blade assembly.

In the embodiments represented here, the lower parts 12 have the shape of a double rectangle, and all the blades are of the same length. Looking at the embodiment shown in FIG. 13, one sees that the lower rectangles 20 of the first blade 1a and second blade 1b carry respectively the outer section 17 of the slot 15 in the first blade and the lower slot 15 of the second blade 1b. As has been seen, these two openings are wide enough to house the assembly formed by the third and fourth blades 1c and 1d. However, it is possible to shorten the first and second blades, by eliminating the lower rectangle 20, although this will reduce the rigidity of the assembly.

An accurate adjustment of the widths of the slots and blades will make it possible to prevent any movement of the blades against each other and to obtain a rigid assembly.

Once the four blades are slotted together, their lower parts 12 are fixed to a rigidifying element, for example, driven into a flat ring 7, which is concentric to the reamer's axis of rotation 19. The flat ring 7 has radial slits 8, made in the inner edge 9 of the said ring in the embodiment represented here. The edges of the lower parts 12 of the blades are engaged in these radial slits 8. Thus fixed on the periphery of their lower parts, the blades form a very rigid assembly. The presence of such a ring 7, however, is not essential in every case: in several applications of the reamer, there is already sufficient rigidity if the widths of the slots are correctly adjusted.

It is also possible to fix the assembled blades by a cylindrical sleeve surrounding the lower parts 12. Similarly, the rigidifying element 7, whether a flat ring, a cylindrical sleeve, or any other form, can be fixed by soldering, gluing or in any other manner. Driving them in remains the simplest method.

This assembly may be made even more rigid, if necessary, by adding a second ring 7 or a second sleeve, of greater diameter, which encircles the blades at a greater distance from the axis of rotation 19.

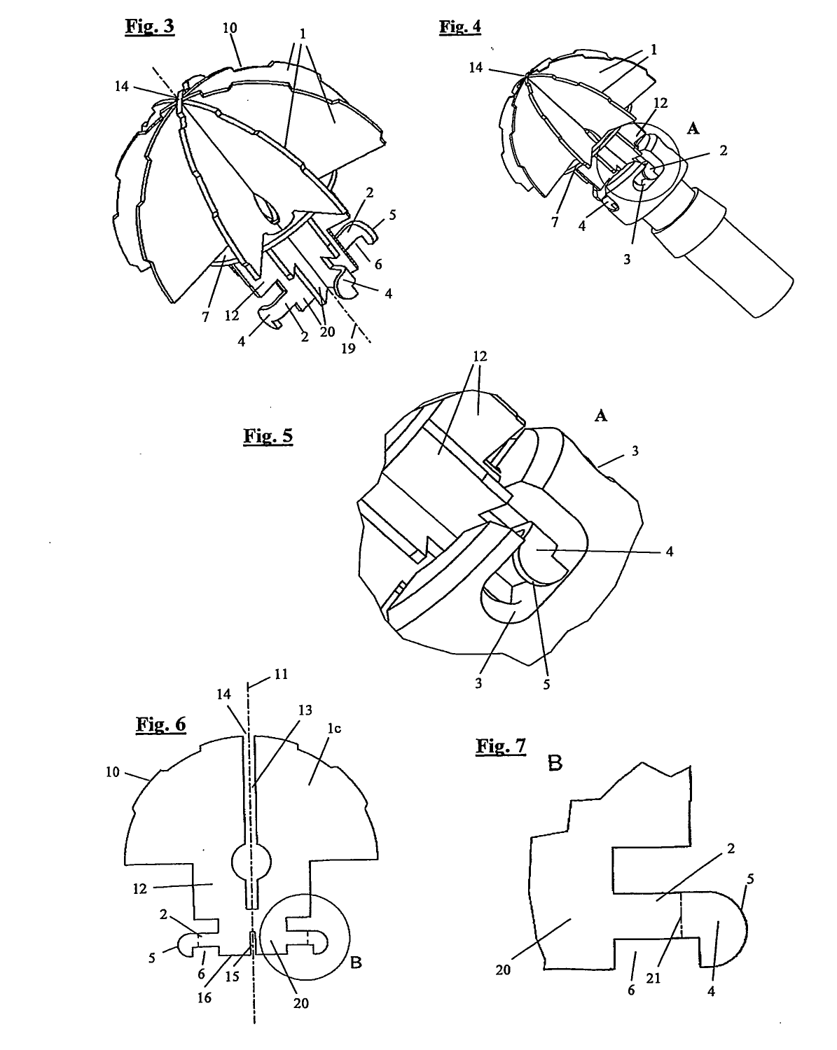

The axis of symmetry 11 of the blades 1 is coincident with the reamer's axis of rotation 19. In order that the blades may be sharpened, where necessary, it is advisable to provide for a hole to be placed on this axis of rotation, as shown in FIGS. 3, 4, 6, 8, 9, 11 and 13.

In the embodiment represented in FIG. 15, the blades are substantially hollow, which increases visibility and reduces the weight. In this embodiment, it is hardly possible to eliminate the bottom 20 of the lower parts 12 of the first and second blades, since this bottom becomes essential to the rigidity of the fixation of the first two blades to the two others.

The third and fourth blades 1c and 1d each have two lateral extensions 2, which project from the lower rectangle 20 of the lower part 12.

These lateral extensions are simply cut from the same sheet as the rest of the blade.

In the embodiment represented in FIGS. 3 to 7, 13 and 15, the lateral extension 2 has a folded part 4, with a rounded end 5. The fold 21 along which the part 4 is folded has a parallel direction to the reamer's axis of rotation 19. The rounded end 5 has a shape that corresponds to that of the bottom of the notch 3 in the cutter spindle on to which the reamer must be fixed. To engage the extension 2 in the notch 3, it suffices to lower the locking part bearing the fingers 22 by sliding it along the shaft of the cutter spindle, thereby releasing the entrance of the notch 3, then to push the reamer down in parallel to its axis of rotation 19, to make it turn around the said axis in order to bring the rounded end 5 to the bottom of the notch 3, to release the locking piece, and thereby cause the finger 22 to lock the extension in the notch.

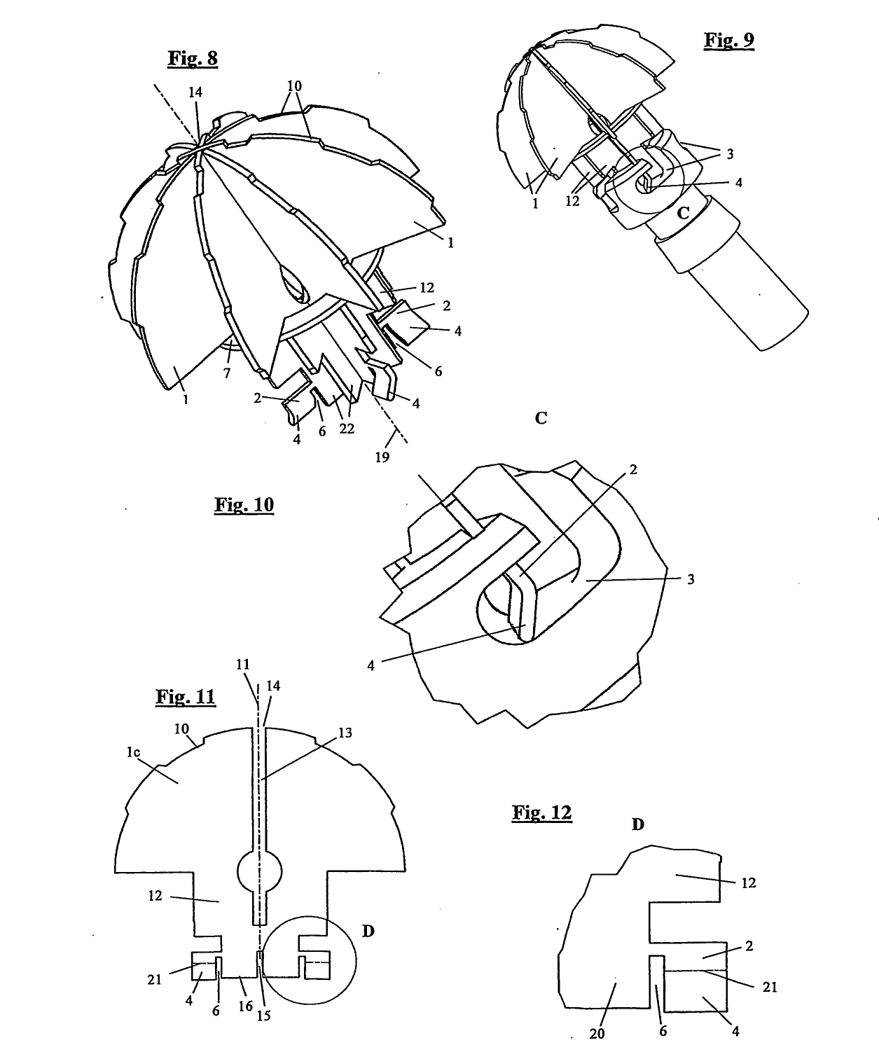

In the embodiment shown in FIGS. 8 to 12, the end of the extension 2 is not rounded, and is folded along a fold 21 which is perpendicular and not parallel to the axis of rotation 19. The folded end 4 of the extension 2 rests on a point against the bottom of the notch 3 on the cutter spindle, while the top of the extension 2 rests on the top of the notch 3. This resting on only two points is sufficient to hold the extension 2 in the notch and thus the reamer in the cutter spindle, provided, of course, that the finger 22 of the cutter spindle, not represented in FIGS. 8 to 10 or in FIGS. 3 to 5, is released from the locked position and closes the notch 3.

A cutout 6 is made in the extension 2, between the lower rectangle 20 and the folded part 4, so as to make the folding easier.

In the drawings, the blades have a semi-circular cutting edge 10 with teeth, which can be used to facilitate reaming depending on the intended purpose. As was seen above, preferably, and as far as possible, the teeth are cut and their angle is obtained in one and the same stamping operation. Similarly, the extensions 2 and their parts 4 can be cut and folded in this same operation.

The thinness of the stamped sheet metal blades, and their position in the plane of the axis of rotation, allows the user to see the part to be reamed, through the blades, even during the operation.

The presence of four blades instead of only two ensures sufficient rigidity to perform operations without any greater risk than with a conventional reamer. Naturally, a different number of blades can be envisaged. Similarly, depending on the cutter spindle for which the reamer is intended, the number of lateral extensions 2 can vary, this obviously being determined by the number of notches on the cutter spindle.

The presence of extensions 2 permits the reamer to be fixed directly in a conventional bayonet-type cutter spindle, without the need for an intermediate tool.

Manufacturing the blades, and their assembly, is easy and inexpensive; they may be disposed of instead of cleaned, which is an onerous operation.

### INDUSTRIAL APPLICABILITY

The reamer and blades of this invention are essentially for use in operations for fitting hip prostheses and may be used on common cutter spindles.

1. Surgical reamer, comprising several flat blades assembled together by means of slots, wherein at least one of the blades has at least one lateral extension cut out from the blade itself and is arranged to be engaged into a notch of a bayonet-type cutter spindle.

2. Reamer according to claim 1, wherein at least either or both one of the slots and sections of said slot or slots has a different width from that of either or both at least one other slot and section of a slot.

3. Reamer according to claim 1, wherein a longitudinal axis of symmetry of the slots is coincident with an axis of rotation of the reamer.

4. Reamer according to claim 1, wherein the lateral extension has a part that is folded in relation to the plane of the blade in which it is cut.

5. Reamer according to claim 4, wherein the folded part has an end that is rounded.

6. Reamer according to claim 1, wherein the lateral extension has a cutout.

7. Reamer according to claim 1, including at least one rigidifying element which partially encircles and holds together the blades making up the reamer.

8. Reamer according to claim 7, wherein the rigidifying element is a flat ring extending in a plane that is perpendicular to an axis of rotation of the reamer and which has radial slits into which the blades are engaged.

9. Reamer according to claim 8, wherein the radial slits are made in the inside edge of the flat ring.

10. Reamer according to claim 1, comprising at least four blades, wherein at least two of which each have two lateral extensions.

11. Reamer according to claim 1, comprising four of said flat blades, the first of which has at least one slot which runs from a lower edge of the blade, wherein a second blade has at least one slot running from a top of a rounded part of the blade, each of said slots having, at least in one section, a width corresponding to a thickness of the blade into which is fitted the other slot, wherein the third blade has at least one upper slot running from a top of said third blade and at least one lower slot running from the lower edge of said third blade, a width of said upper slot being greater than the width of said lower slot, and wherein the fourth blade has at least one slot running from a top of said blade, said slot comprising at least one outer section and one inner section, a width of said outer section being greater than a width of said inner section, a width of a lower slot of the third blade to a thickness of the fourth blade, a width of an inner section of the slot of the fourth blade corresponding to a thickness of the third blade, and the width of the upper slot of the third blade and the width of the outer section of the fourth blade being the same such that, once fitted together, the first two blades can be inserted into said upper slot of the third blade and into the outer section of the fourth blade, said third and fourth blades being themselves fitted together.

12. Reamer according to claim 11, wherein the slot in the first blade has at least two sections of different widths, the outer section being wider than the inner section.

13. Reamer according to claim 11, wherein the second blade has at least one lower slot running from a lower edge thereof, said lower slot being wider than the slot which runs from the top of the blade.

14. Blade for reamer according to claim 1, comprising a rounded upper part, and a narrower lower part in which is arranged a lateral extension.

15. Blade according to claim 14, comprising at least one slot with sections of different widths.

16. Blade according to claim 14, comprising at least one slot running from the top of the rounded upper part, or from a lower edge of the blade.

17. Blade according to claim 16, comprising at least one of an upper slot running from the top of the rounded upper part and a lower slot running from a lower edge, the width of the upper slot being different from the width of the lower slot.

18. Blade according to claim 14, comprising either or both an upper slot running from the top of the rounded upper part and a lower slot running from a lower edge, at least one of the said upper or lower slots comprising at least one outer section and one inner section, a width of the outer section being greater than a width of the inner section.
